(19) United States
(12) Patent Application Publication
(10) Pub. No.: US 2001/0007855 A1
(43) Pub. Date: Jul. 12, 2001

(54) SEMI-SYNTHETIC STUDIES TOWARD DIDEMNIN ANALOGUES

(76) Inventors: KENNETH L. RINEHART, URBANA, IL (US); ALEXANDRA J. KATAUSKAS, LINCOLN, IL (US)

Correspondence Address: BANNER & WITCOFF, 28 STATE STREET, 28TH FLOOR, BOSTON, MA, 02109

(21) Appl. No.: 09/284,906

(22) PCT Filed: Oct. 24, 1997
(86) PCT No.: PCT/US97/19210

§ 371 Date: None

(51) Int. Cl. A61K 38/00; C07K 5/00; C07K 7/00; C07K 16/00; C07K 17/00; A61K 38/12

(52) U.S. Cl. 514/10; 530/317

(57) ABSTRACT

Disclosed are semi-synthetic methods for the preparation of Didemnin analogs. The compounds of this type are illustrated in Formula (I).

[chemical structure]

### SUMMARY OF THE INVENTION

The syntheses of several didemnin derivatives, including didemnin M (1) as well as pyroglutaminyl didemnin B (2), have been performed. Didemnin M, one of the most active of the didemnins, contains pyroglutamate, glutamine, lactyl, and proline groups in its side chain, while pyroglutaminyl didemnin B contains only a pyroglutaminyl unit in addition to the lactyl and prolyl residues. Glutaminyl derivatives (3-5) were also synthesized in the process of producing didemnin M.

[chemical structure]

The retrosynthetic disconnections which formed the basis of a plan for preparation of the side chain of didemnin M are shown in Equation 1. Disconnection of the ester function between lactic acid and L-glutamine would give two units: a dipeptide, unit 7, comprised of pyroglutamate and glutamine; and unit 8, comprised of lactic acid and proline.

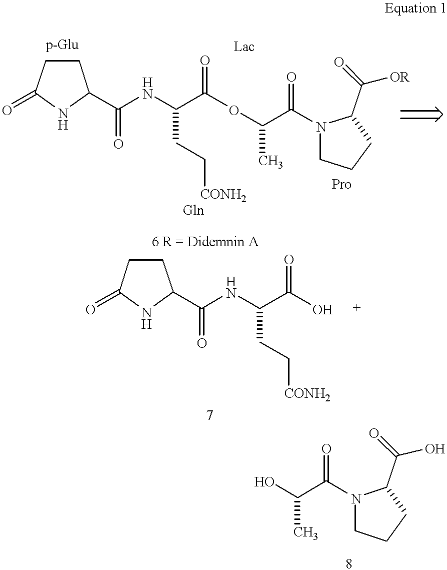

A mixed anhydride from L-pyroglutamic acid 9 and pivaloyl chloride was coupled with L-glutamine t-butyl ester 10 followed by acidic workup to yield L-pyroglutamyl-L-glutamine 7 (Equation 2). This dipeptide was purified by reversed phase HPLC using a gradient system of acetonitrile/H₂O.

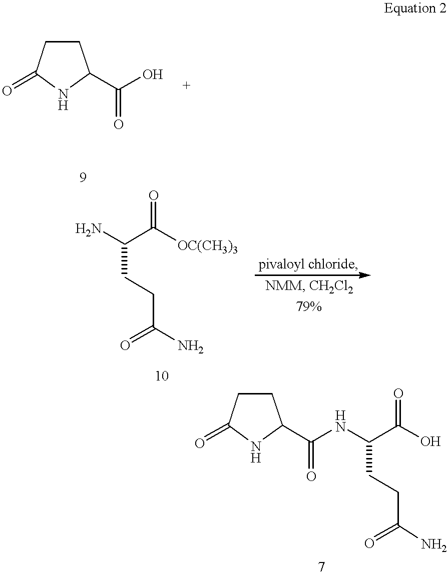

The synthesis of compound 8 began with protection of (S)-ethyl lactate, 11, as the benzyloxy derivative 12. Hydrolysis provided the acid 13 which was coupled with L-proline phenacyl ester to afford compound 14. Treatment with a solution of zinc in acetic acid afforded 8 (Scheme I).

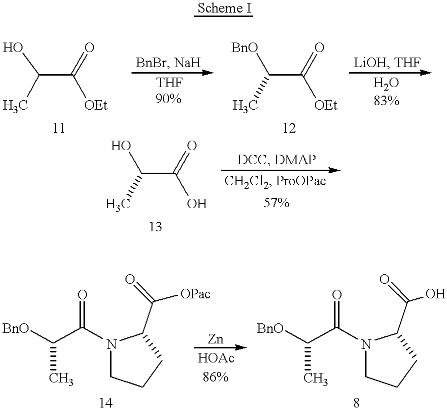

Didemnin M was synthesized by a three step scheme involving a coupling reaction of benzyllactylproline, 8, with didemnin A to give the protected derivative 15 followed by hydrogenation to yield didemnin B. The final step involved coupling of the pyroglutaminylglutamine unit, 7, with didemnin B. This was carried out using a variety of techniques with the most efficient being the mixed anhydride method (Scheme II). Purification was performed using HPLC with an acetonitrile/H₂O gradient system.

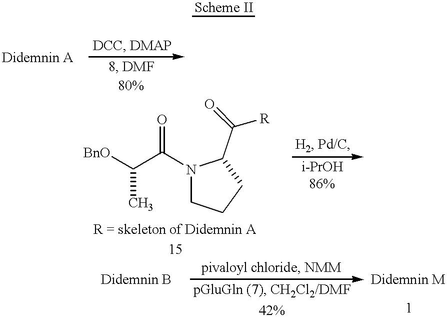

A second approach toward the synthesis of didemnin M involved protecting L-glutamine, 16, as the benzyloxycarbonyl derivative, 17, followed by coupling with didemnin B using DCC. During this coupling procedure, two glutaminyl derivatives were produced, 18, bore a glutaminyl residue at only the lactyl residue while the second, 19, contained two glutaminyl residues, one on the lactyl unit and the second on the isostatine unit. These derivatives were separated via reversed phase HPLC, then hydrogenated to provide the deprotected compounds 3 and 4 (Scheme III).

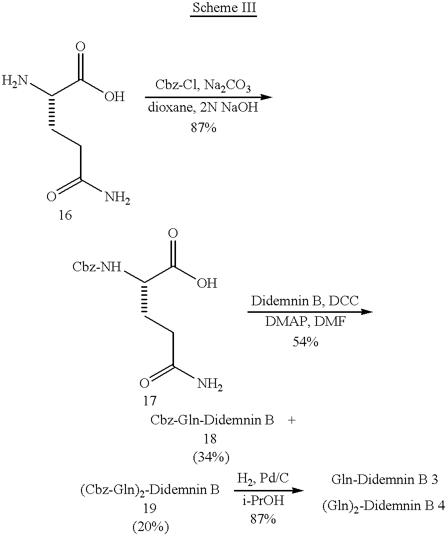

A different attempt at deprotection of the benzyloxycarbonyl derivative 18 provided yet another glutaminyl didemnin analogue. This analogue was formed upon treatment of 18 with hydrogen bromide in acetic acid. It appears as though an acetyl unit was added to the isostatine residue to provide compound 5 (Equation 3). These two compounds appear to be easily separable by reversed phase HPLC. This deprotection technique also proved to be useful with the dibenzyloxycarbonylglutaminyl derivative of didemnin B. 19.

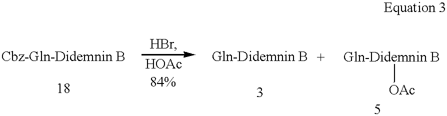

Pyroglutamic acid was protected as the benzyloxycarbonyl derivative (20) which was then coupled with glutaminyldidemnin B (3) using DCC to provide the protected form of didemnin M (21). Deprotection via hydrogenation afforded didemnin M (1) (Scheme IV). Purification via reversed phase HPLC provided the desired compound.

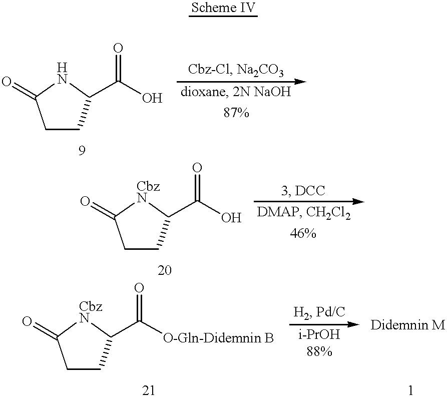

Another interesting analogue of didemnin is pyroglutaminyldidemnin B (2). The synthesis of 2 was accomplished by coupling 20 to didemnin B using EDC to provide Cbz-pyroglutaminyl didemnin B 22. Removal of the protecting group was accomplished using hydrogenation in the presence of a palladium catalyst to afford 2. Purification via reversed phase HPLC, using an acetonitrile/water gradient system, provided the pure compound (Equation 4).

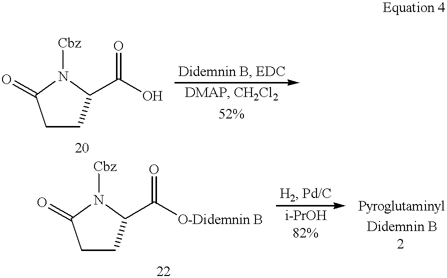

Dehydrodidemnin B was synthesized by first coupling Boc-L-proline (23) to didemnin A using EDC as the coupling agent. The Boc protecting group was removed upon treatment with acid and the resulting compound (25) was coupled with pyruvic acid to provide dehydrodidemnin B (Scheme V). The compound was purified via reversed phase HPLC using a gradient system of acetonitrile/H₂O.

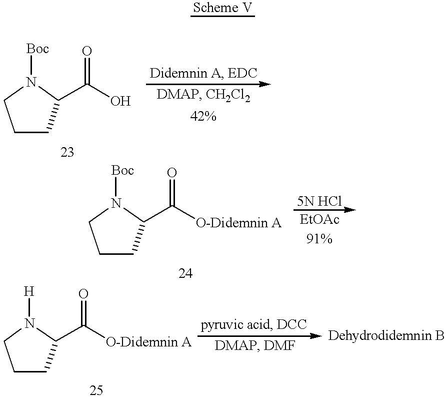

### DETAILED DESCRIPTION OF THE PREFERRED EMBODIMENTS

General Experimental Procedures. ¹NMR spectra were recorded on Varian XL-200, General Electric QE-300, Varian XL-400,and General Electric QN-500 spectrometers. ¹H Chemical shifts are referenced in CDCl₃ and methanol-d4 to residual CHCl₃ (7.26 ppm) and CD₂HOD (3.34 ppm). Electron impact (EI) mass spectra were recorded on a Finnigan MAT CH-5 DF spectrometer. High resolution (HRFAB) and fast atom bombardment (FAB) mass spectra were recorded on a VG ZAB-SE mass spectrometer operating in the FAB mode using magic bullet matrix.²⁷ Microanalytical results were obtained from the School of Chemical Sciences Microanalytical Laboratory. Infrared (IR) spectra were obtained on an IR/32 FTIR spectrophotometer. Solid samples were analyzed as chloroform solutions in sodium chloride cells. Liquids or oils were analyzed as neat films between sodium chloride plates.

Optical rotations (in degrees) were measured with a DIP 360 or a DIP 370 digital polarimeter with an Na lamp (589 nm) using a 5-×0.35-cm (1.0 mL) cell. Melting points were determined on a capillary melting point apparatus and are not corrected. Normal phase column chromatography was performed using Merck-kieselgel silica gel (70-230 mesh). Fuji-Davison C18 gel (100-200 mesh) was used for reversed phase column chromatography. All solvents were spectral grade. Analytical thin layer chromatography was performed on precoated plates (Merck, F-254 indicator). These plates were developed by various methods including exposure to ninhydrin, iodine, and UV light (254 nm). HPLC was performed with a Waters 990 instrument and an Econosil C₁₈ column (Alltech/Applied Science) and a Phenomenex C₁₈ column.

THF was distilled from sodium benzophenone ketyl and CH₂Cl₂ from P₂O₅. Dimethylformamide (DMF), triethylamine (Et₃N), and N-methylmorpholine (NMM) were distilled from calcium hydride and stored over KOH pellets. Pyridine was distilled from KOH and stored over molecular sieves. Other solvents used in reactions were reagent grade without purification. Di-tert-butyl dicarbonate [(Boc)₂O], dicyclohexylcarbodiimide (DCC), 1-(3-dimethylaminopropyl)-3-ethylcarbodiimide hydrochloride (EDCI), dimethylaminopyridine (DMAP), 1-hydroxybenzotriazole (HOBT), L-glutamine, L-pyroglutamine, and L-proline were obtained from the Aldrich Chemical Company. All reactions requiring anhydrous conditions were performed under an atmosphere of nitrogen.

Pyroglutaminylglutamine (7). Pyroglutamic acid (0.11 g, 0.84 mmol) was dissolved in DMF (2.09 mL) and the solution was cooled to −20° C. N-Methylmorpholine (0.19 mL) and pivaloyl chloride (0.10 mL) were added to the solution and stirring continued at −20° C. for 5 h. At this time, a solution of glutamine t-butyl ester (0.20 g, 0.84 mmol) in DMF (0.42 mL) and N-methylmorpholine (92 mL) was added dropwise. Stirring was continued for 48 h, and the solution was allowed to warm to room temperature, then poured into H₂O and extracted with EtOAc. The EtOAc layer was washed with 1N HCl and H₂O, then dried (Na₂SO₄), and the solvent was carefully removed below 40° C. A white solid was isolated. Recrystallization from ether/petroleum ether provided 7 as a white crystalline material (0.17 g, 79%); FABMS 258.1 (M+H); HRFABMS calcd for C₁₀H₁₅N₃O₅ (M+H) 258.1090, found 258.1091.

Ethyl (S)-O-Benzyllactate (12). To a solution of ethyl (S)-lactate (2.36 g, 20.0 mmol) in THF (7.80 mL) was added sodium hydride (60% dispersion, 0.94 g, 24.0 mmol) portionwise, with cooling. Benzyl bromide (2.60 mL, 22.0 mmol) was then added via a dropping funnel. The reaction was allowed to stand at room temperature for 24 h. Ethyl acetate (70 mL) was slowly added to the reaction mixture, followed by water, to destroy the excess sodium hydride. The solution was then evaporated to dryness and the oily residue was partitioned between ether (30 mL) and water (60 mL). The ether layer was washed with aqueous sodium bicarbonate (5 mL) and brine. The solution was dried over sodium sulfate and the solvent evaporated to give an oily residue which crystallized overnight. Recrystallization of the crude product gave the compound as a white crystalline material (3.01 g, 72%); FABMS m/z 209.1 (M+H), 181.2 (M—C₂H₄).

O-Benzyllactic acid (13). To a cold solution of 12 (0.31 g, 1.49 mmol) in THF (14.9 mL) was added, dropwise, a cold 0.2 M lithium hydroxide solution (14.9 mL) during 10-min. Stirring continued for 3 h at ambient temperature, then the solution was concentrated to half its volume and washed with ether (2×15 mL). The combined ether layers were extracted with saturated NaHCO₃ (10 mL), and the aqueous layers were combined and acidified to pH 4 with 1N potassium hydrogen sulfate. The acidified aqueous layer was extracted with ether (3×50 mL) and the combined ether extracts were dried (Na₂SO₄), filtered, and concentrated under reduced pressure, providing the corresponding acid an oil, which was used directly in the next step (0.21 g, 80%); ¹H NMR (500 MHz, CDCl₃) δ 1.46 (3H, d), 4.05 (1H, q), 4.55 (2H, dd), 7.31 (5H, s), 11.36 (1H, s); FABMS 219.0 (M+K), 203.1 (M+Na), 181.2 (M+H), HRFABMS calcd for C₁₀H₁₂NaO₃ (M+Na) 203.0684, found 203.0686; m/z calcd for C₁₀H₁₃O₃ (M+H) 181.0865, found 181.0864.

Boc-L-Proline Phenacyl Ester. Boc-proline (1.00 g, 4.65 mmol) was dissolved in ethyl acetate (29.4 mL), triethylamine (0.46 g, 0.63 mL) and phenacyl bromide (0.93 g, 4.68 mmol) were added and, within a few minutes, a precipitate formed. The mixture was stirred overnight, water and ether were added and the two layers separated. The organic layer was washed with 0.1N HCl, saturated sodium bicarbonate, and brine, then dried over MgSO₄. Evaporation of the solvent provided the desired compound (1.27 g, 83%); FABMS 334.2 (M+H), 234.1 (M+2H−Boc), 667.3 (2M+H); HRFABMS calcd for C₁₈H₂₃NO₅ (M+H) 334.1654, found 334.1665.

L-Proline phenacyl ester. Boc-L-proline phenacyl ester (0.29 g, 0.87 mmol) was dissolved in EtOAc (25 mL) and a steady current of HCl was passed through the solution for approximately 40 min, when TLC analysis showed the deprotection to be complete. The solvent was evaporated to provide a white crystalline material. Recrystallization from petroleum ether gave clear crystals (0.19 g, 94%); FABMS 234.2 (M+H), 467.2 (2M+H); HRFABMS calcd for C₁₃H₁₆NO₃ (M+H) 234.1130, found 234.1129.

L-O-Benzyllactyl-proline Phenacyl Ester (14). Proline phenacyl ester (0.19 g, 0.83 mmol) in CH₂Cl₂, DMAP (0.10 g, 0.83 mmol) and DCC (0.19 g, 0.96 mmol) were added at 0° C. to a solution of 13 (0.15 g, 0.83 mmol). The solution was allowed to warm to room temperature and stirred for 12 h. Dicyclohexylurea was filtered and washed with ethyl acetate. The filtrate and washings were combined and washed with 10% citric acid, 5% sodium bicarbonate and water, dried over MgSO₄ and concentrated. The crude residue was purified by flash chromatography eluting with hexane and ethyl acetate (4:1) to obtain the product (0.19 g, 57%) as an orange oil; FABMS 396.2 (M+H); HRFABMS calcd for C₂₃H₂₆NO₅ (M+H) 396.1811, found 396.1812.

L-O-Benzyllactyl-proline (8). Compound 14 (0.19 g, 0.48 mmol) was treated with Zn (0.96 g) in AcOH/H₂O (70:30), the mixture was allowed to stir at rt overnight, Zn was filtered off using celite, and the solution was partitioned between ether and water. The organic layer was separated and dried over Na₂SO₄ to afford the desired compound (0.11 g, 86%); FABMS 278.1 (M+H).

O-Benzyldidemnin B (15). L-O-Benzyllactyl-proline (33.0 mg, 0.13 mmol) in DMF (3 mL), DMAP (0.6 mg) and DCC (26.0 mg, 0.13 mmol) were added at 0° C. to a solution of didemnin A (39.7 mg, 0.42 mmol). The solution was allowed to warm to room temperature and stirred for 12 h, dicyclohexylurea was filtered and washed with ethyl acetate. The filtrate and washings were combined and washed with 10% citric acid, 5% sodium bicarbonate and water, and the extracts were dried over MgSO₄ and concentrated. The crude residue was purified by reversed phase HPLC using a gradient system of acetonitrile/H₂O to provide the compound as a yellow powder (40.5 mg, 80%); ¹H NMR (500 MHz, CDCl₃), see Supplementary Material, S-1; FABMS 1241.2 (M+K), 1226.1 (M+Na), 1203.1 (M+H), see Supplementary Material, S-2; HRFABMS calcd for C₆₄H₉₆N₇O₁₅ (M+H) 1202.6964, found 1202.6964.

Didemnin B. Protected didemnin B (15, 40.5 mg, 33.7 mmol) was dissolved in isopropyl alcohol (5 mL), palladium on carbon (10%) catalyst (37.4 mg) was added and the solution was hydrogenated at room temperature and atmospheric pressure for 3 h, when TLC showed the reaction to be complete. The catalyst was filtered over celite and the solvent was evaporated to provide the desired compound as a white powder. Reversed phase HPLC (acetonitrile/H₂O gradient system) revealed the compound to be pure, see Supplementary Material, S-3 (32.1 mg, 86%); ¹H NMR (500 MHz, CDCl₃), see Supplementary Material, S-4; FABMS 1134.5 (M+Na), 1112.5 (M+H), Supplementary Material, S-5; HRFABMS calcd for C₅₇H₉₀N₇O₁₅ (M+H) 1112.6495, found 1112.6491.

Pyroglutaminyl-glutaminyl-didemnin B [Didemnin M (1)]. Pyroglutaminylglutamine (3.42 mg, 14.4 μmol) was dissolved in DMF (36.0 μL) and the solution was cooled to −20 ° C. N-Methylmorpholine (3.27 μL) and pivaloyl chloride (1.72 μL) were added to the solution and stirring continued at −20 ° C. for 5 h, when a solution of didemnin B (16.0 mg, 14.4 μmol) in CH₂Cl₂ (7.23 μL) and N-methylmorpholine (1.59 μL) was added dropwise. Stirring continued for 48 h, then the solution was allowed to warm to room temperature, and the mixture was poured into H₂O and extracted with EtOAc. The EtOAc layer was washed with 1N HCl and H₂O, dried (Na₂SO₄), and solvent was carefully removed below 40° C. Reversed phase HPLC using a gradient system of acetonitrile/H₂O afforded the desired compound, see Supplementary Material, S-6 (8.1 mg, 79%); ¹H NMR (500 MHz, CDCl₃), see Supplementary Material, S-7; FABMS m/z 1389.5 (M+K), 1374.5 (M+Na), 1351.6 (M+H), see Supplementary Material, S-8; HRFABMS m/z calcd for C₆₇H₁₀₃N₁₀O₁₉ (M+H) 1351.7401, found 1351.7406.

N-Benzyloxycarbonyl-L-glutamine (17). Glutamine (1.84 g. 12.62 mmol) was dissolved in 1N NaOH (12.58 mL) and the solution was cooled to 0° C. and stirred for 30 min, when Na₂CO₃ (3.30 g) and benzyl chloroformate (4.38 mL) in dioxane (19.30 mL) were gradually added, in equal portions. Stirring continued at 0° C. for 1 h, then the solution was allowed to stir overnight at room temperature and was extracted with ethyl ether (2×20 mL). The aqueous solution was acidified with 2N HCl to pH 5 and extracted with ethyl acetate (3×50 mL), which was dried over sodium sulfate, and evaporated to give an oil which crystallized overnight. Recrystallization of the crude product gave a white crystalline material (3.07 g. 87%); FABMS 319.1 (M+K), 281.1 (M+H).

N-Benzyloxycarbonyl-L-glutaminyl-didemnin B (18). To a solution of Cbz-glutamine (0.14 g, 0.55 mmol) in dry DMF (2.50 mL), DMAP (0.6 mg) and DCC (20.6 mg, 0.11 mmol) were added at 20° C. with stirring. Stirring continued at room temperature for 2 h and a solution of didemnin B (23.0 mg, 20.6 μmol) in DMF (2.50 mL) was added with stirring. The solution was stirred at room temperature for 24 h, diluted with CH₂Cl₂ and washed with 5% NaHCO₃ and water to neutral pH. The solution was dried (Na₂SO₄) and evaporated to give a white solid which was purified by reversed phase HPLC using a gradient system of acetonitrile/water, see Supplementary Material, S-9 (51.3 mg, 34%); ¹H NMR (500 MHz, CDCl₃), see Supplementary Material, S-10; FABMS 1374.6 (M+H), see Supplementary Material, S-11; HRFABMS calcd for C₇₀H₁₀₄N₉O₁₉ (M+H) 1374.7448, found 1374.7446. A second derivative was also obtained from HPLC purification (see Supplementary Material, S-12) and was found to be di-(benzyloxycarbonyl)glutaminyl-didemnin B (36.0 mg, 20%); ¹H NMR (500 MHz, CDCl₃), see Supplementary Material, S-13; FABMS 1637.2 (M+H), see Supplementary Material, S-14; HRFABMS calcd for C₈₃H₁₁₈N₁₁O₂₃ (M+H) 1636.8402, found 1636.8401.

Glutaminyl-didemnin B (3). Compound 18 (25.1 mg, 18.2 μmol) was dissolved in isopropyl alcohol (1.00 mL) and 10% Pd/C catalyst (0.99 mg) was added. The solution was hydrogenated for 3 h. The catalyst was removed by filtration over celite and solvent was removed to afford 3 which was purified by reversed phase HPLC using a gradient system of acetonitrile/water (see Supplementary Material, S-15) (19.6 mg, 87%); ¹H NMR (500 MHz, CDCl₃), see Supplementary Material, S-16; FABMS 1278.5 (M+K), 1262.6 (M+Na), 1240.7 (M+H), see Supplementary Material, S-17; HRFABMS calcd for C₆₂H₁₀₆N₁₁O₁₉ (M+H) 1240.7081, found 1240.7076.

Glutaminyl-glutaminyl-didemnin B (4). The procedure was identical to that described above for 3. Compound 4 was also prepared by treatment of 19 with hydrogen bromide in acetic acid; FABMS 1368.7 (M+H), see Supplementary Material, S-18; HRFABMS calcd for C₆₇H₁₀₆N₁₁O₁₉ (M+H) 1368.7666, found 1368.7680.

N-Benzyloxycarbonyl-L-pyroglutamine (20). L-Pyroglutamine (2.02 g, 13.83 mmol) was dissolved in 1 N NaOH (13.84 mL) and the solution was cooled to 0° C. After 30 min stirring, Na₂CO₃ (3.63 g) and benzyl chloroformate (4.82 mL) in dioxane (21.23 mL) were gradually added, in equal portions. Stirring was continued at 0° C. for 1 h, then the solution was stirred overnight at room temperature and extracted with ethyl ether (2×20 mL). The aqueous solution was acidified with 2N HCl to pH 5, extracted with ethyl acetate (3×50 mL), dried over sodium sulfate, and evaporated to give an oil which crystallized overnight. Recrystallization of the crude product gave white crystalline material (2.86 g, 87%); FABMS 240.1 (M+H).

L-(N-Benzyloxycarbonyl-pyroglutaminyl)-L-glutaminyl-didemnin B (21). To a solution of Cbz-pyroglutamine (10.2 mg, 38.7 μmol) in dry DMF (0.18 mL), DMAP (0.22 mg) and DCC (7.59 mg, 7.74 μmol) were added at 20° C. with stirring. Stirring continued at room temperature for 2 h and a solution of didemnin B (9.60 mg, 7.74 μmol) in DMF (2.50 ml) was added with stirring. The solution was stirred at room temperature for 24 h. The solution was diluted with CH₂Cl₂ and washed with 5% NaHCO₃ and water to neutral pH. The solution was dried (Na₂SO₄) and solvent evaporated to give 21 as a white solid. The compound was purified by reversed phase HPLC using a gradient system of acetonitrile/water (see Supplementary Material, S-19) (5.19 mg, 46%); ¹H NMR (500 MHz, CDCl₃), see Supplementary Material, S-20; FABMS 1524.2 (M+K), 1509.1 (M+Na), 1485.8 (M+H), see Supplementary Material, S-21; HRFABMS calcd for C₇₅H₁₀₉N₁₀O₂₁ (M+H) 1485.7769, found 1485.7765.

L-Pyroglutaminyl-L-glutaminyl-didemnin B [Didemnin M (1)]. Compound 21 (2.12 mg, 1.40 μmol) was dissolved in isopropyl alcohol (1.00 mL) and 10% Pd/C catalyst (9.90 μg) was added. The solution was hydrogenated for 3 h, catalyst was removed by filtration over celite and solvent was removed to afford the desired compound. The compound was purified by reversed phase HPLC using a gradient system of acetonitrile/water (see Supplementary Material, S-6) (1.66 mg, 88%); ¹H NMR (500 MHz, CDCl₃), see Supplementary Material, S-7; FABMS 1389.5 (M+K), 1374.5 (M+Na), 1351.6 (M+H), see Supplementary Material S-8; HRFABMS calcd for C₆₇H₁₀₃N₁₀O₁₉ (M+H) 1351.7401, found 1351.7406.

N-Benzyloxycarbonyl-L-pyroglutaminyl-didemnin B (22). DMAP (0.48 mg) and EDC (16.5 mg, 88.0 μmol) were added at 20° C. with stirring to compound 20 (0.11 g, 0.44 mmol) in dry CH₂Cl₂ (2.00 mL). Stirring continued at room temperature for 2 h and a solution of didemnin B (9.20 mg, 8.24 μmol) in CH₂Cl₂ (2.00 mL) was added with stirring. The solution was stirred at room temperature for 24 h, diluted with CH₂Cl₂ and washed with 5% NaHCO₃ and water to neutral pH. The solution was dried (Na₂SO₄) and the solvent evaporated to give the compound as a white solid. The compound was purified by reversed phase HPLC using a gradient system of acetonitrile/water (5.70 mg, 52%); FABMS 1356.7 (M+H), see Supplementary Material, S-22; HRFABMS calcd for C₇₀H₁₀₂N₉O₁₈ (M+H) 1356.7343, found 1356.7335.

L-Pyroglutaminyl-didemnin B (2). Compound 22 (5.70 mg, 4.28 μmol) was dissolved in isopropyl alcohol (0.5 mL) and 10% Pd/C catalyst (0.25 mg) was added. The solution was hydrogenated for 5 h, catalyst was removed by filtration, and the solvent was removed to afford 22, which was purified by reversed phase HPLC using a gradient system of acetonitrile/water, see Supplementary Material, S-23 (4.28 mg, 82%); ¹H NMR (500 MHz, CDCl₃), see Supplementary Material, S-24; FABMS 1223.7 (M+H), see Supplementary Material, S-25; HRFABMS calcd for C₆₂H₉₅N₈O₁₇ (M+H) 1223.6815, found 1223.6811.

Boc-L-prolyl-didemnin A (24). DMAP (0.75 mg) and EDC (11.5 mg, 60.0 mmol) were added at 20° C. with stirring to Boc-L-proline (23) (25.0 mg, 0.12 mmol) in dry CH₂Cl₂ (2.00 mL). Stirring continued at room temperature for 2 h and a solution of didemnin B (44.4 mg, 40.0 mmol) in CH₂Cl₂ (2.00 mL) was added with stirring. The solution was stirred at room temperature for 24 h. The solution was diluted with CH₂Cl₂ and washed with 5% NaHCO₃ solution and water to neutral pH. The solution was dried (Na₂SO₄) and the solvent evaporated to give the compound as a white solid (17.5 mg, 42%); FABMS 1140.6. (M+H), 1040.6 (M+2H−Boc).

L-Prolyl-didemnin B (25). Compound 24 (15.1 mg, 13.2 μmol) was dissolved in 5N HCl in ethyl acetate. After 3 h stirring at room temperature, TLC analysis showed the deprotection to be complete. The solvent was evaporated to provide a white crystalline material (12.5 mg, 91%); FABMS 1040.6 (M+H), see Supplementary Material, S-26.

Dehydrodidemnin B. DMAP (0.16 mg) and DCC (2.62 mg, 12.8 μmol) were added at 20° C. with stirring to a solution of pyruvic acid (2.61 mg, 29.7 μmol) in dry DMF (0.10 mL). Stirring continued at room temperature for 2 h and a solution of prolyl-didemnin A (10.3 mg, 9.90 μmol) in DMF (0.40 mL) was added with stirring. The solution was stirred at room temperature for 24 h, diluted with CH₂Cl₂ and washed with 5% NaHCO₃ solution and water to neutral pH, then dried (Na₂SO₄) and the solvent evaporated to give the product as a white solid. The compound was purified by reversed phase HPLC using a gradient system of acetonitrile/water (see Supplementary Material, S-27) to give a white powdery substance; ¹H NMR (500 MHz, CDCl₃), see Supplementary Material, S-28; FABMS 1110.6 (M+H), see Supplementary Material, S-29; HRFABMS calcd for C₅₇H₈₈N₇O₁₅ (M+H) 1110.6338, found 1110.6334.

TABLE I
Antiviral Activities of Didemnins^{a} (# - New Compounds)

| | | HSV/CV-1 | | |
|---|---|---|---|---|
| | | | | Ac- |
| | Compound | ng/mL | Cytotoxocity^{b} | tivity^{c} |
| # | Gln-Didemnin B | 100 | 16 | ? |
| | | 50 | 16 | ? |
| | | 20 | 16 | ? |
| | | 10 | 0 | +++ |
| # | Cbz-Gln-Didemnin B (161) | 100 | 0 | + |
| | | 50 | 0 | + |
| | | 20 | 0 | + |
| | | 10 | 0 | − |
| | Didemnin M (5) | 100 | 16 | ? |
| | | 50 | 16 | ? |
| | | 20 | 0 | +++ |
| | | 10 | 0 | + |
| # | pGlu-Didemnin B (39) | 100 | 16 | ? |
| | | 50 | 16 | ? |
| | | 20 | 0 | +++ |
| | | 10 | 0 | + |
| # | Cbz-pGlu-Didemnin B (145) | 100 | 0 | + |
| | | 50 | 0 | + |
| | | 20 | 0 | + |
| | | 10 | 0 | − |
| # | Gln[GlnIst²]-Didemnin B (160) | 100 | 0 | +++ |
| | | 50 | 0 | + |
| | | 20 | 0 | + |
| | | 10 | 0 | + |
| # | Cbz-Gln[Cbz-GlnIst²]DB (162) | 100 | 0 | +++ |
| | | 50 | 0 | + |
| | | 20 | 0 | + |
| | | 10 | 0 | + |
| | O-Bu-Didemnin B (140) | 100 | 16 | ? |
| | | 50 | 9 | + |
| | | 20 | 8 | + |
| | | 10 | 0 | + |
| | Didemnin (B) (2) | 100 | 16 | ? |
| | | 50 | 0 | +++ |
| | | 20 | 0 | +++ |
| | | 10 | 0 | + |
| | Dehydrodidemnin B (6) | 100 | 16 | ? |
| | | 50 | 16 | ? |
| | | 20 | 0 | +++ |
| | | 10 | 0 | + |
| | Didemnin A (1) | 100 | 0 | + |
| | | 50 | 0 | + |
| | | 20 | 0 | + |
| | | 10 | 0 | − |

FOOTNOTES: ^{a}Performed by Dr. G. R. Wilson in this laboratory; ^{b}0 (least toxic) to 16 (toxic); ^{c}+++ = complete inhibition; ++ = strong inhibition; + = moderate inhibition; − = no inhibition.

TABLE III
T/C (% of Control, Life Extension) vs. P388

Murine Leukemia in Mice

| | | |
|---|---|---|
| #Gln-DB | T/C | Dose, mg/kg |

| | |
|---|---|
| 185 | 1 |
| 171 | 0.05 |
| 152 | 0.025 |

TABLE II
Cytotoxicity of Didemnins^{a} # = New Compounds

| | | Dose (ng/mL) | | | | |
|---|---|---|---|---|---|---|
| | | 250 | 25 | 2.5 | 0.25 | IC₅₀ |
| | Compounds | Inhibition (%) | | | | (ng/mL) |
| # | Gln-Didemnin B (141) | 100 | 100 | 100 | 94 | 0.1 |
| # | PGlu-Didemnin B (39) | 100 | 100 | 100 | 94 | 0.1 |
| | Dehydrodidemnin B (6) | 100 | 100 | 100 | 95 | 0.2 |
| | Didemnin M (6) | 100 | 100 | 100 | 94 | 0.8 |
| | Didemnin B (2) | 100 | 100 | 40 | 0 | 7 |
| | O-Bu-didemlnin B (140) | 100 | 97 | 0 | NT^{b} | 10 |
| | Prolyl-didemnin A (43) | 100 | 99 | 40 | 30 | 12 |
| # | Cbz-Gln-didemnin B (161) | 100 | 87 | 0 | 0 | 25 |
| # | (Cbz-Gln)₂-didemnin B (162) | 99 | 87 | 0 | 0 | 50 |
| # | (Gln)₂-didemnin B (160) | 100 | 87 | 0 | 0 | 50 |
| # | Cbz-pGlu-didemnin B (145) | 100 | 70 | 0 | 0 | 50 |
| | Didemnin A (1) | 100 | 70 | 0 | 0 | 75 |
| | Boc-Pro-didemnin A (158) | 100 | 55 | 0 | 0 | 85 |

^{a}Performed by Dr. G. R. Wilson in this laboratory. ^{b}NT = not tested.

TABLE III
T/C (% of Control, Life Extension) vs. P388

Murine Leukemia in Mice

| | T/C | Dose, mg/Kg |
|---|---|---|
| # Gln-DB | 185 | 1 |
| 171 | 0.05 | |
| 152 | 0.025 | |

### BRIEF DESCRIPTION OF THE DRAWINGS

FIG. 1 is a ¹H NMR spectrum of O-Benzyldidemnin B (15).

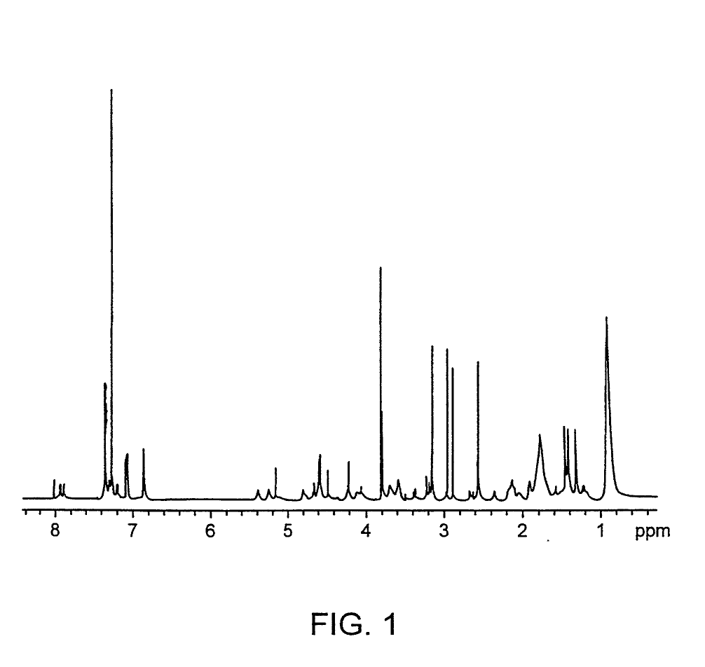

FIG. 2 is a LRFAB mass spectrum of O-Benzyldidemnin B (15).

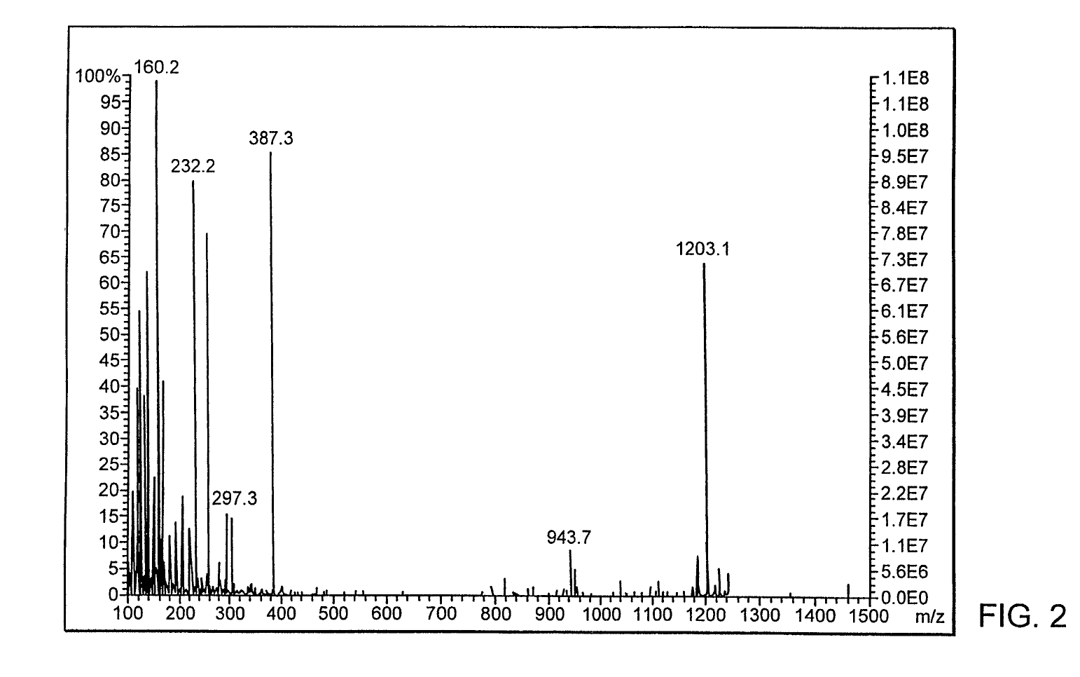

FIG. 3 is a RPHPLC trace of Didemnin B.

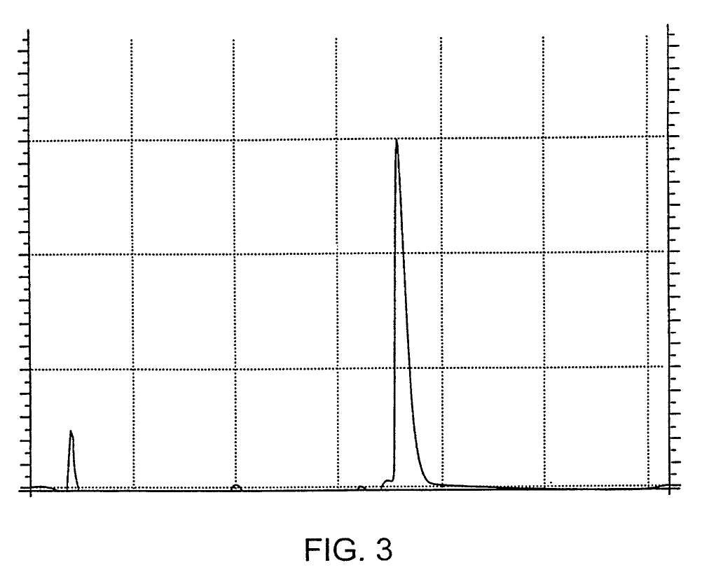

FIG. 4 is a ¹H NMR spectrum of Didemnin B.

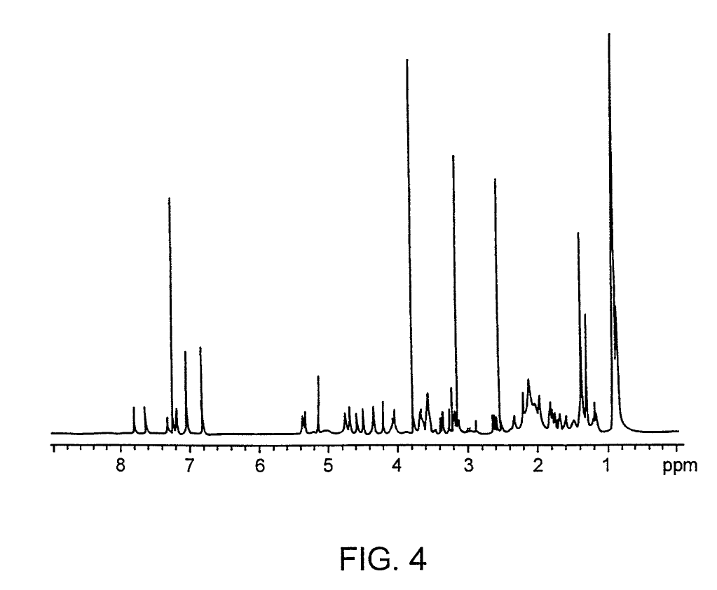

FIG. 5 is a LRFAB mass spectrum of O-Benzyldidemnin B (15).

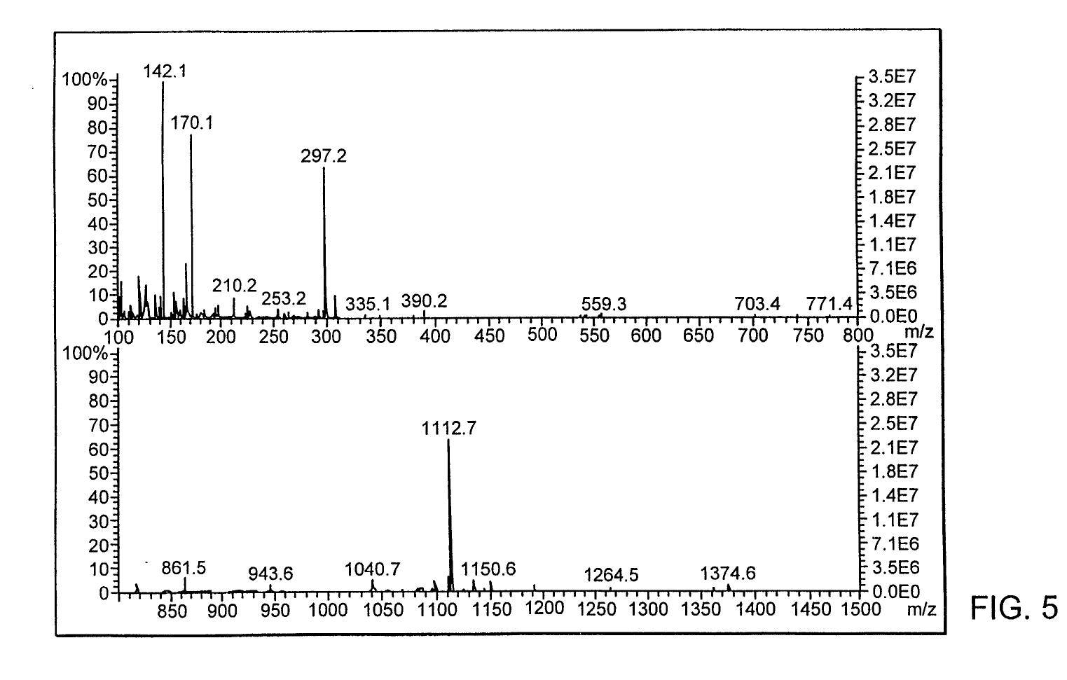

FIG. 6 is a RPHPLC trace of Didemnin M (1).

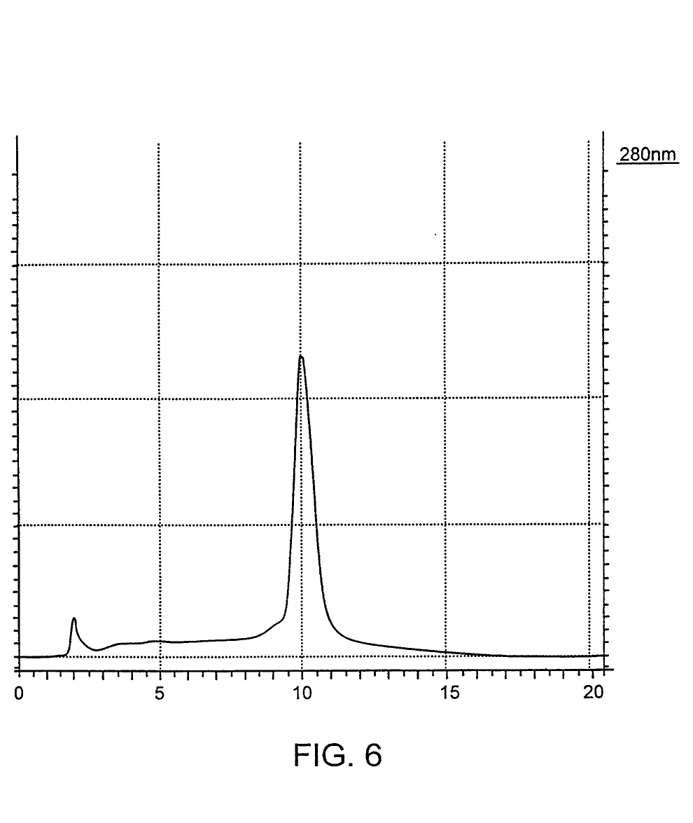

FIG. 7 is a ¹H NMR spectrum of Didemnin M (1).

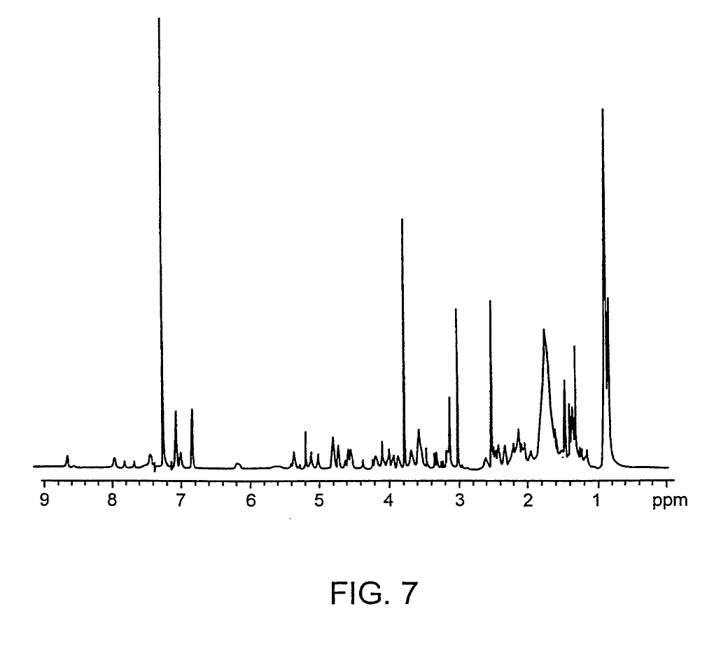

FIG. 8 is a LRFAB mass spectrum of Didemnin M (1).

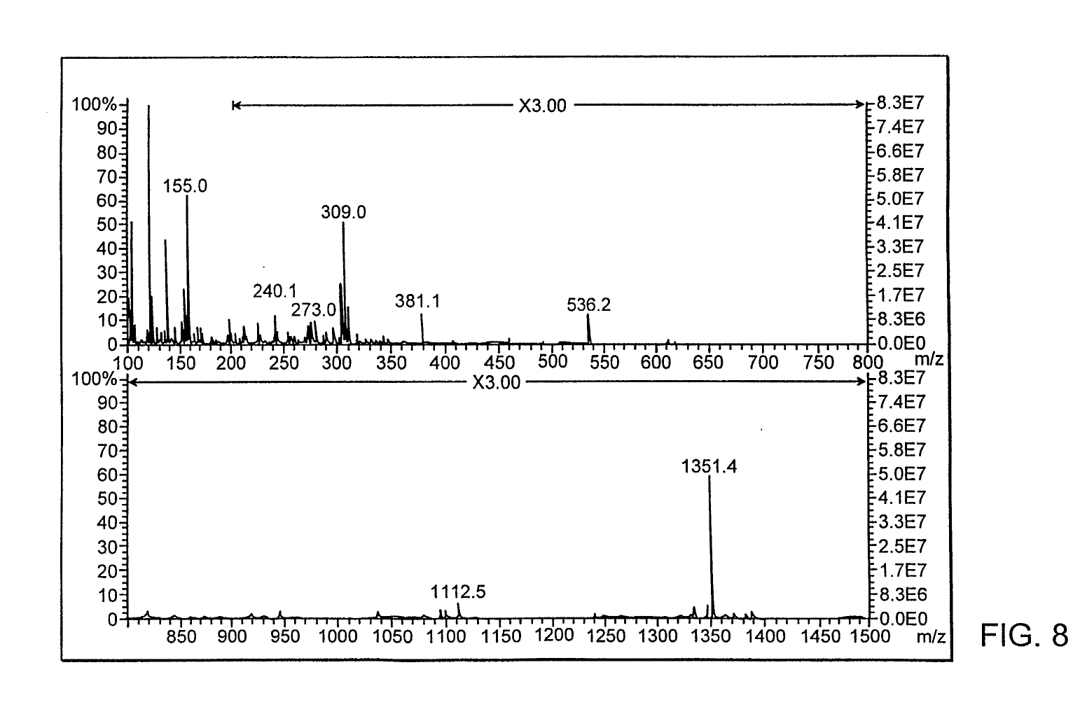

FIG. 9 is a RPHPLC trace of Benzyloxycarbonyl-L-Glutaminyldidemnin B (18).

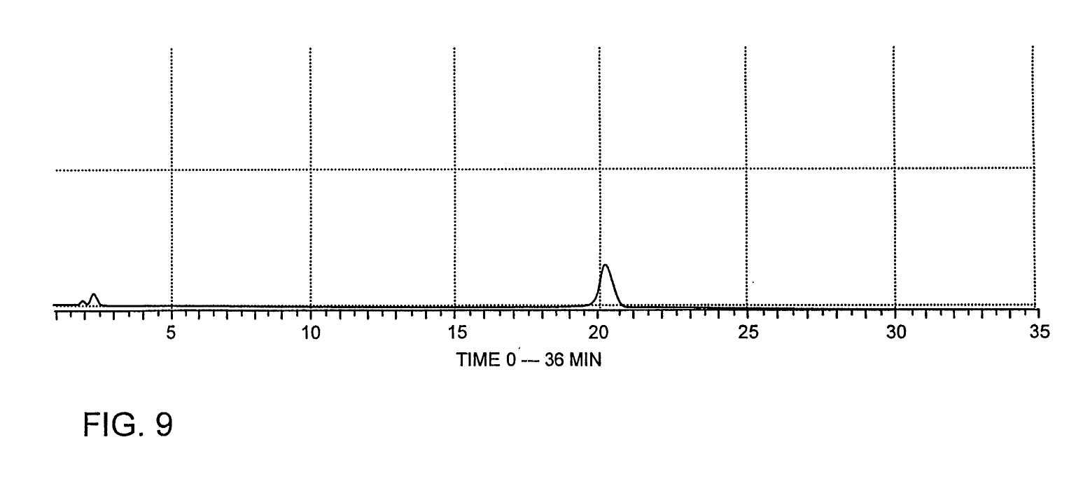

FIG. 10 is a ¹H NMR spectrum of Benzyloxycarbonyl-L-Glutaminyldidemnin B (18).

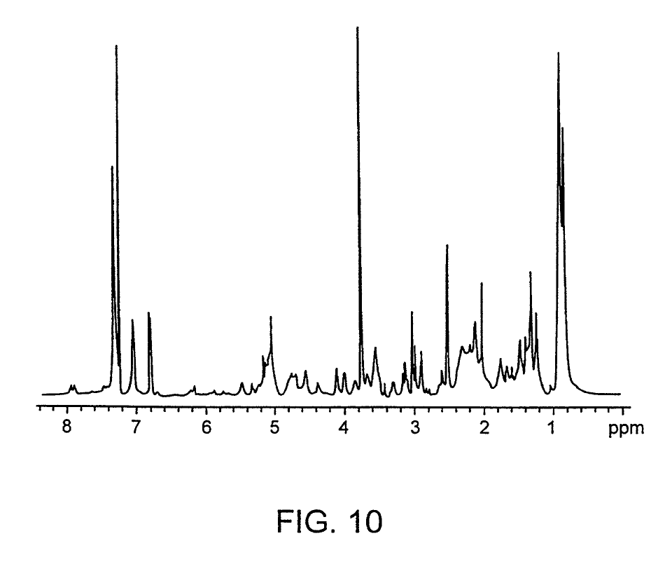

FIG. 11 is a LRFAB mass spectrum of Benzyloxycarbonyl-L-Glutaminyldidemnin B (18).

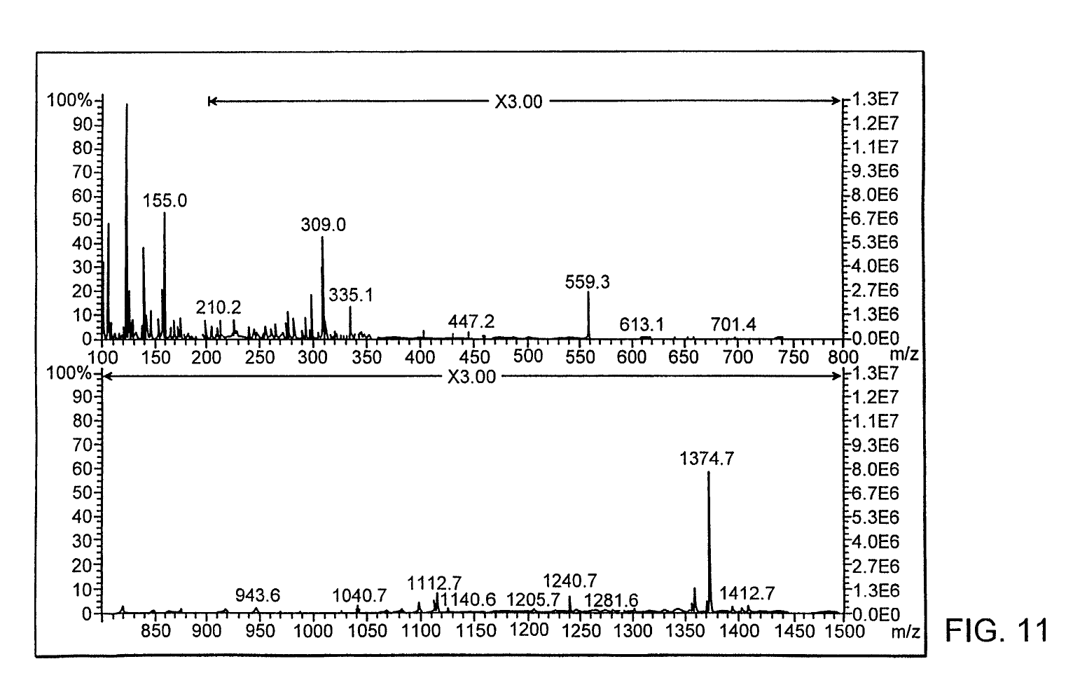

FIG. 12 is a RPHPLC trace of (Benzyloxycarbonyl-L-Glutaminy)₂-Didemnin M B(19).

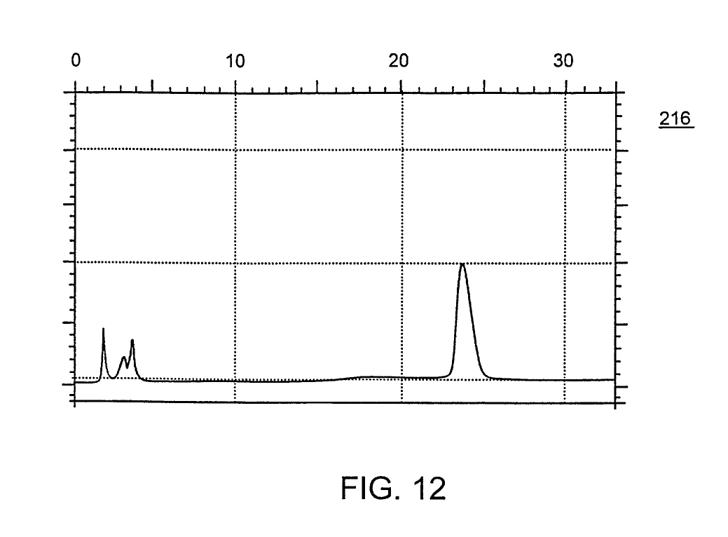

FIG. 13 is a ¹H NMR spectrum of (Benzyloxycarbonyl-L-Glutaminy)₂. Didemnin M B(19).

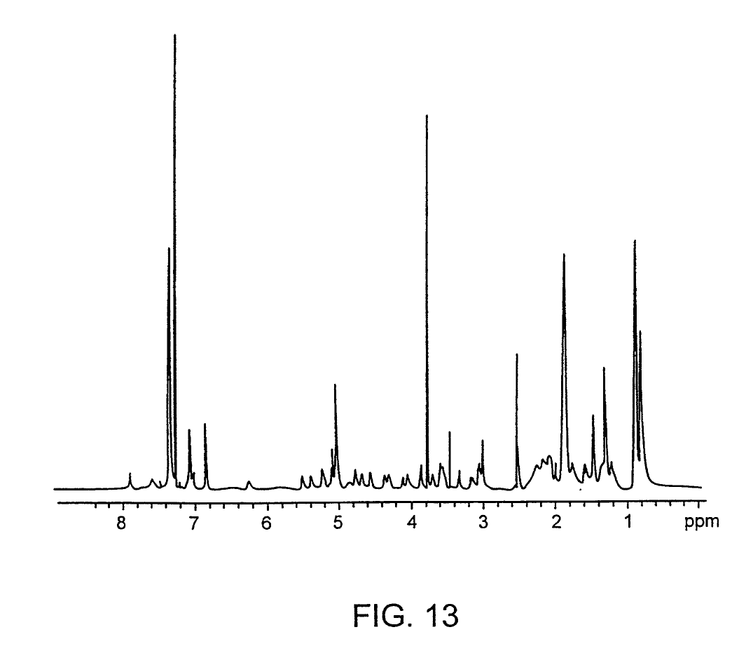

FIG. 14 is a LRFAB mass spectrum of (Benzyloxy-carbonyl-L-Glutaminy)₂ Didemnin M B(19).

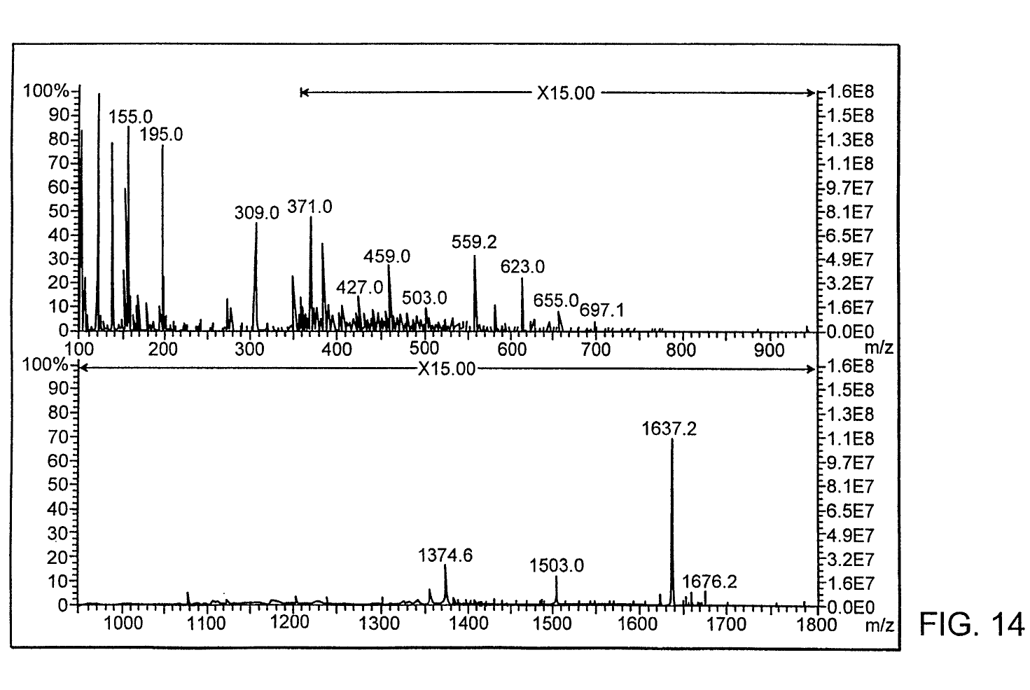

FIG. 15 is a RPHPLC trace of Glutaminyldidemnin B (3).

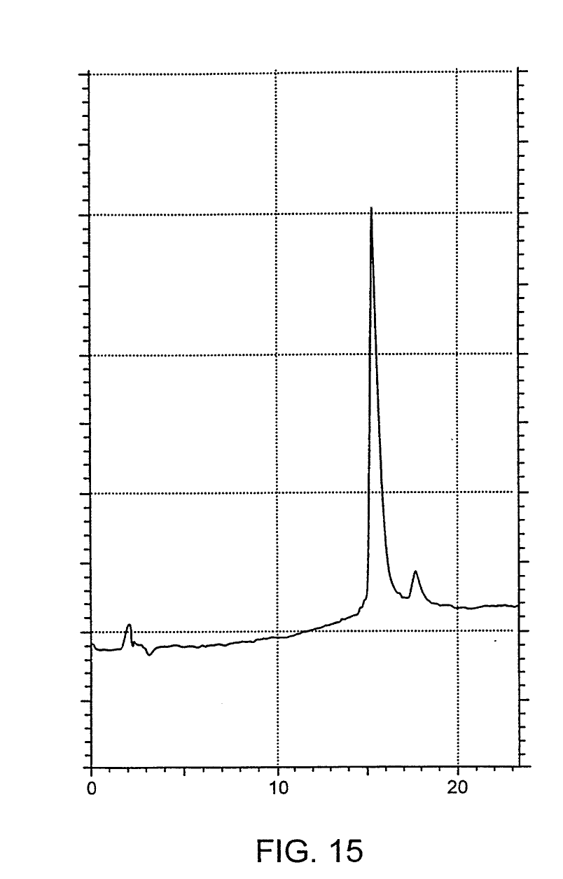

FIG. 16 is a ¹H NMR spectrum of Glutaminyl-didemnin B (3).

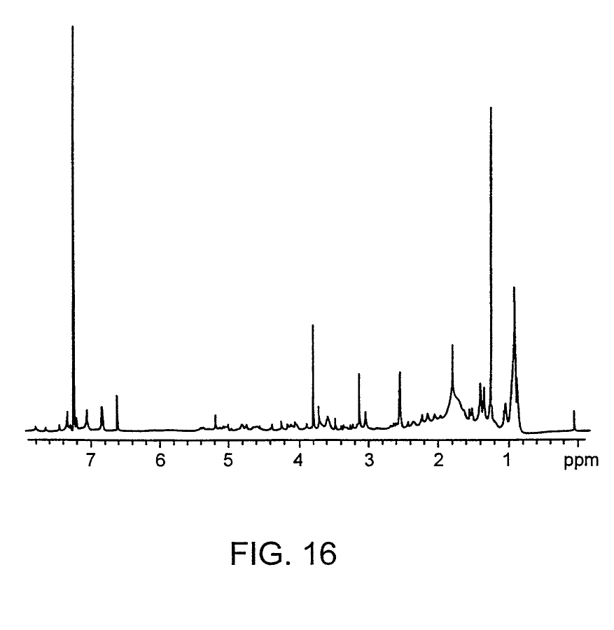

FIG. 17 is a LRFAB mass spectrum of Glutaminyldidemnin B (3).

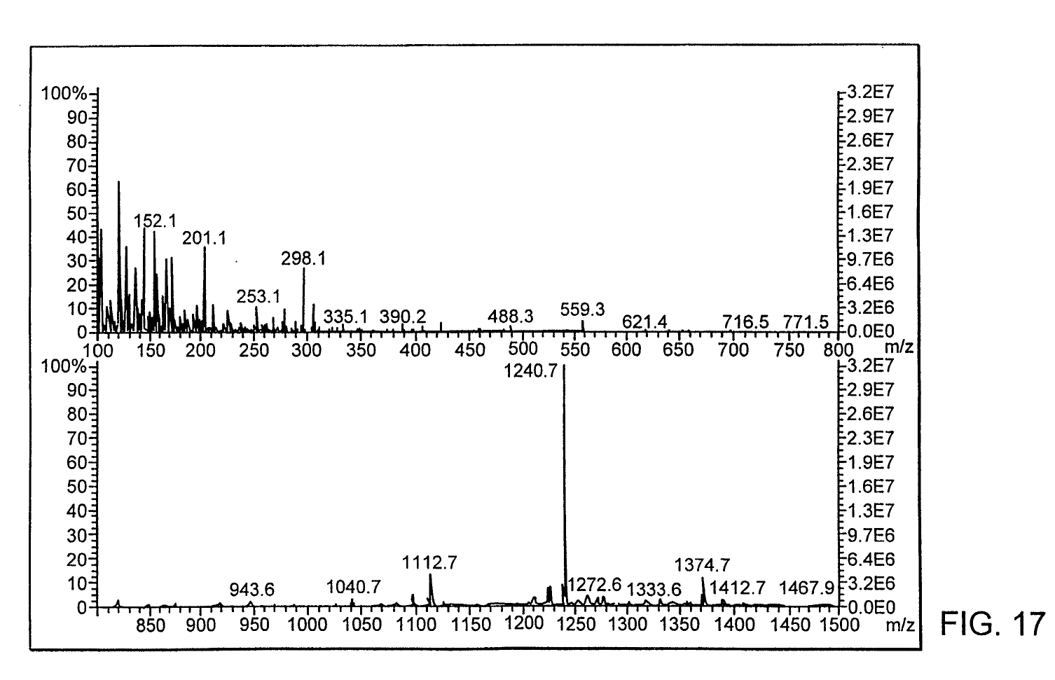

FIG. 18 is a LRFAB mass spectrum of Diglutaminyldidemnin B (4).

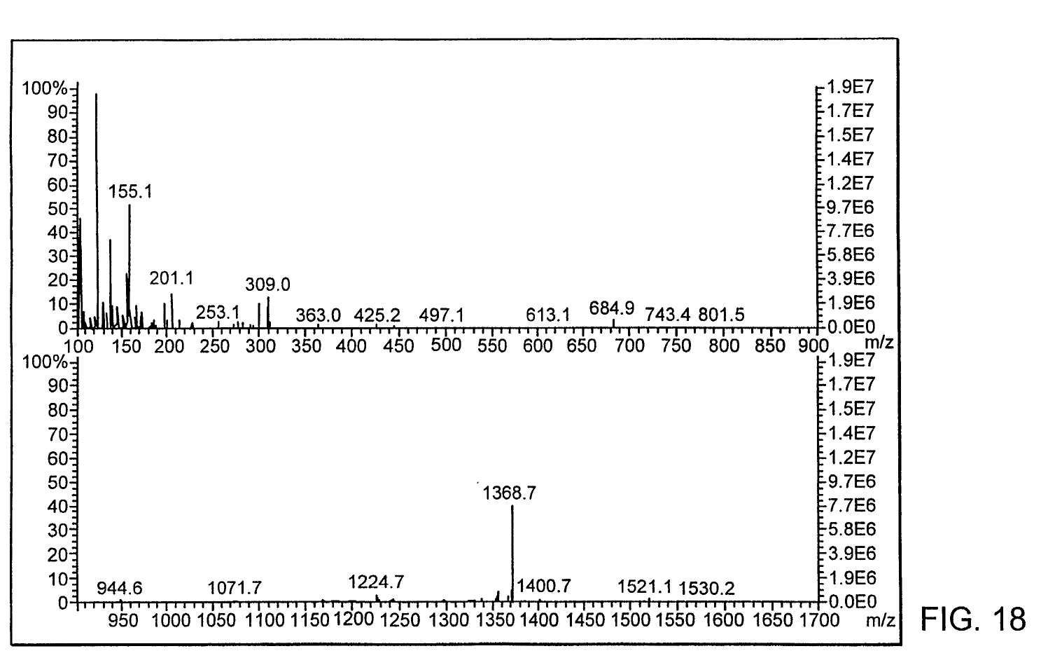

FIG. 19 is a RPHPLC trace of Benzyloxycarbonyldidemnin M (21).

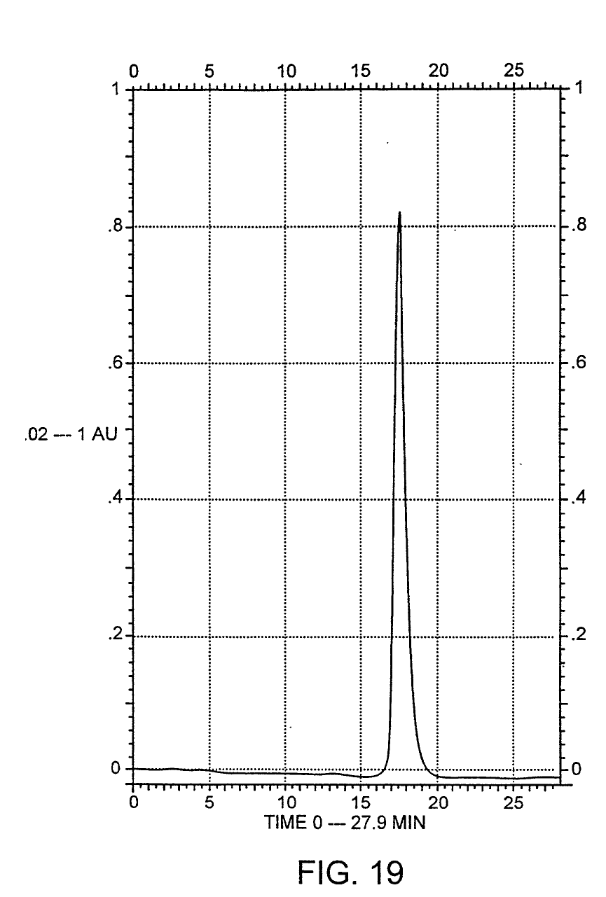

FIG. 20 is a ¹H NMR spectrum of Benzyloxycarbonyldidemnin M (21).

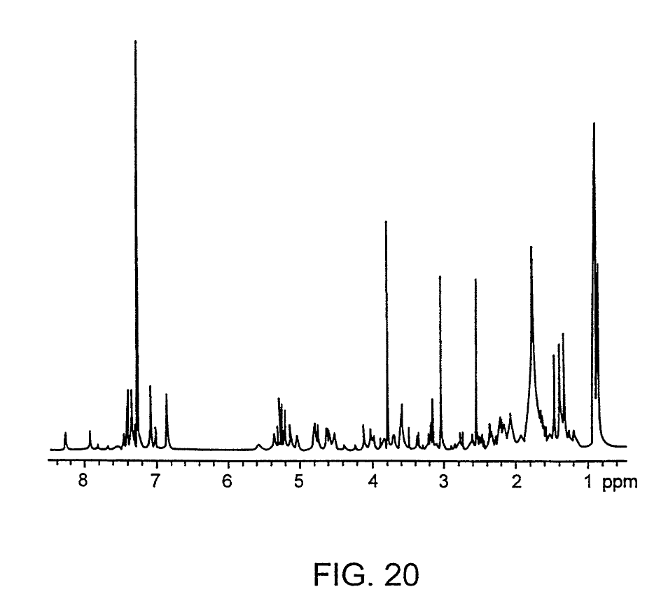

FIG. 21 is a LRFAB mass spectrum of Benzyloxycarbonyldidemnin M (21).

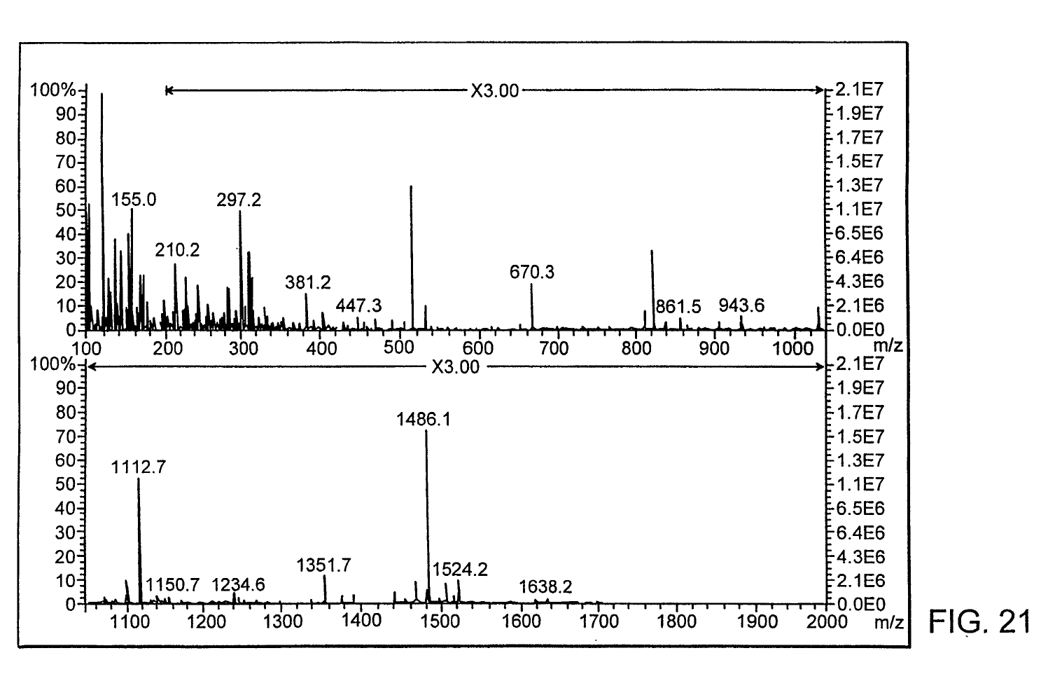

FIG. 22 is a LRFAB mass spectrum of Benzyloxycarbony-L-Pyroglutaminyldidemnin B (22).

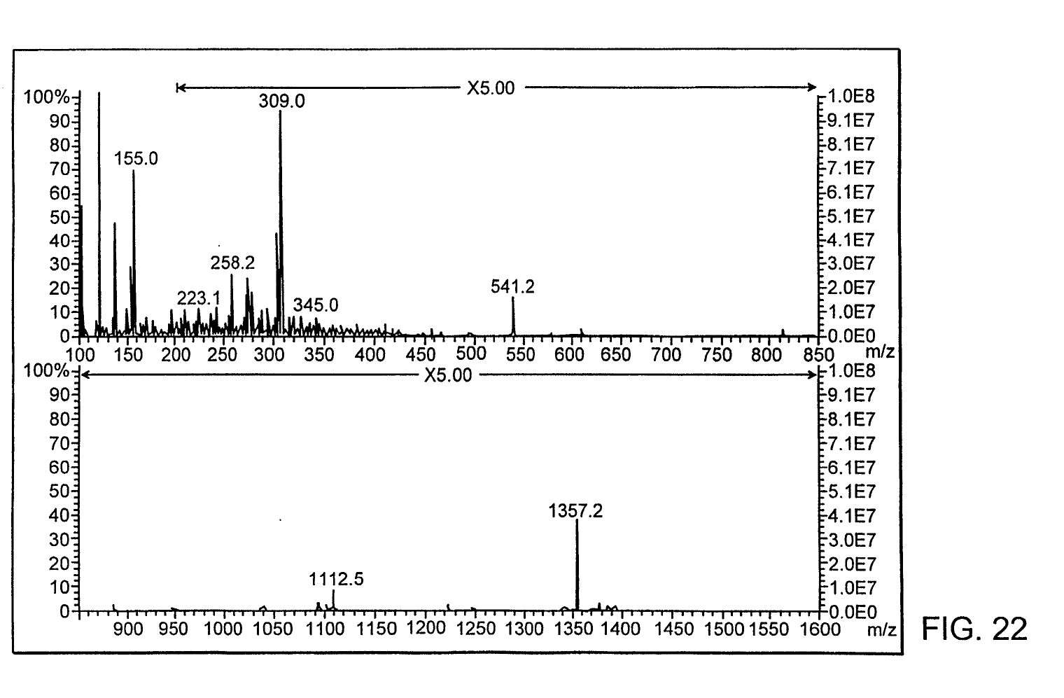

FIG. 23 is a RPHPLC trace of Pyroglutaminyldidemnin B (23).

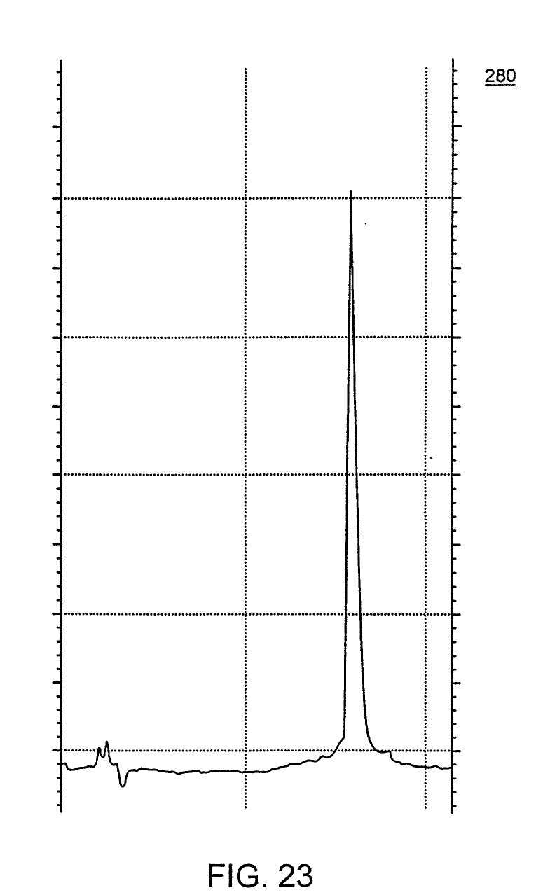

FIG. 24 is a ¹H NMR spectrum of Pyroglutaminyldidemnin B (23).

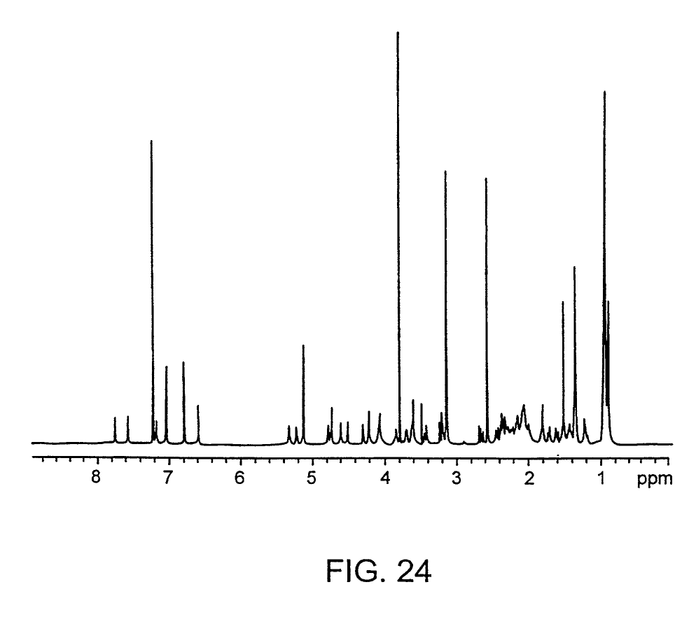

FIG. 25 is a LRFAB mass spectrum of Pyroglutaminyldidemnin B (23).

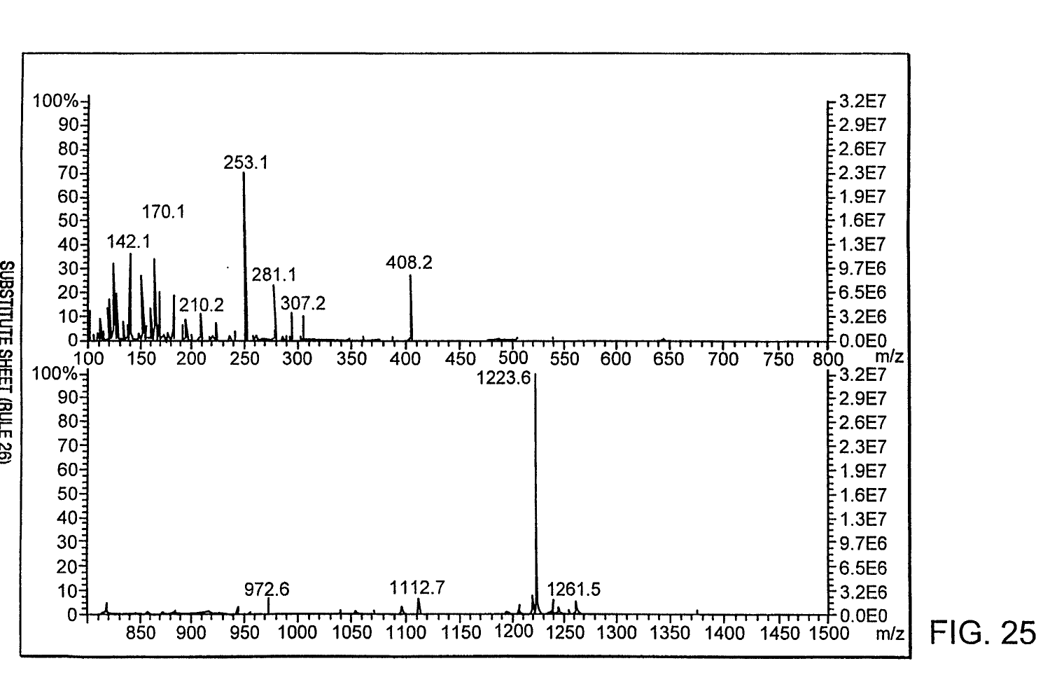

FIG. 26 is a LRFAB mass spectrum of Prolydidemnin A (25).

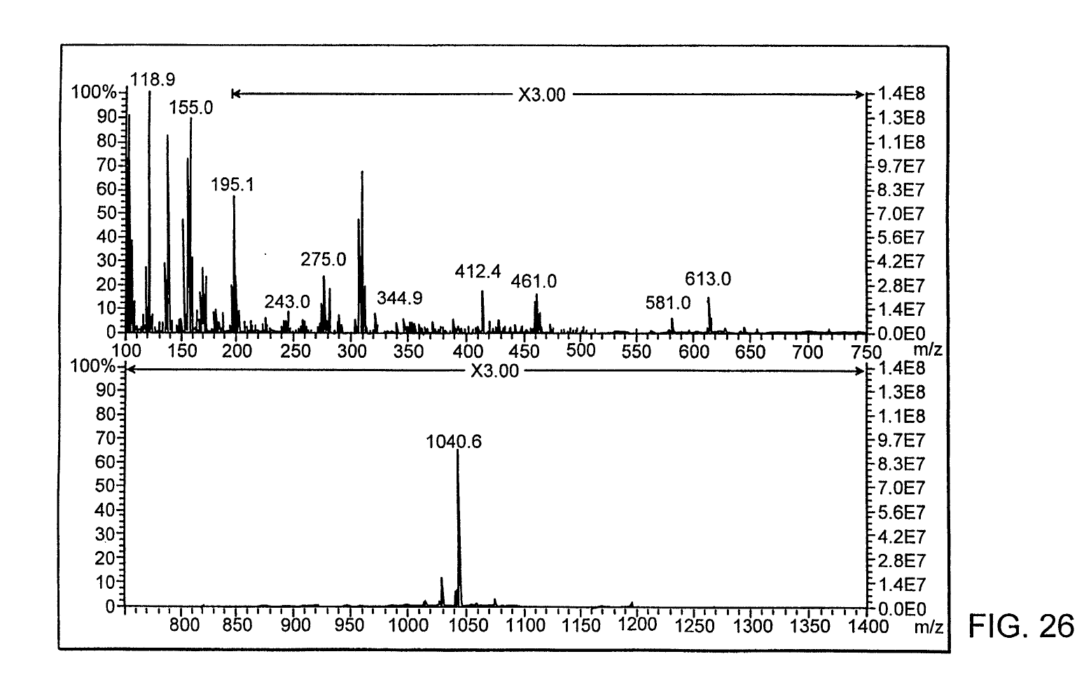

FIG. 27 is a RPHPLC trace of Dehydrodidemnin B.

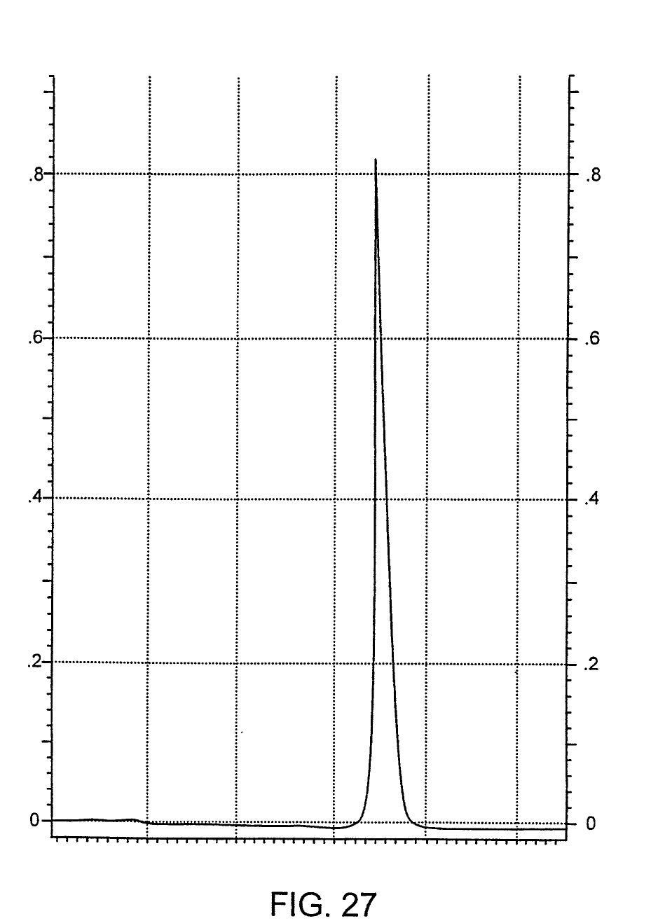

FIG. 28 is a ¹H NMR spectrum of Dehydrodidemnin B.

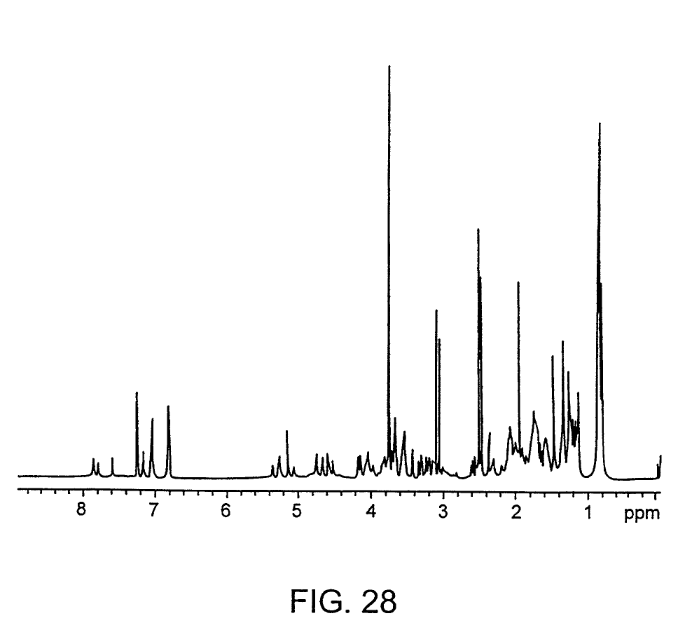

FIG. 29 is a LRFAB mass spectrum of Dehydrodidemnin B.

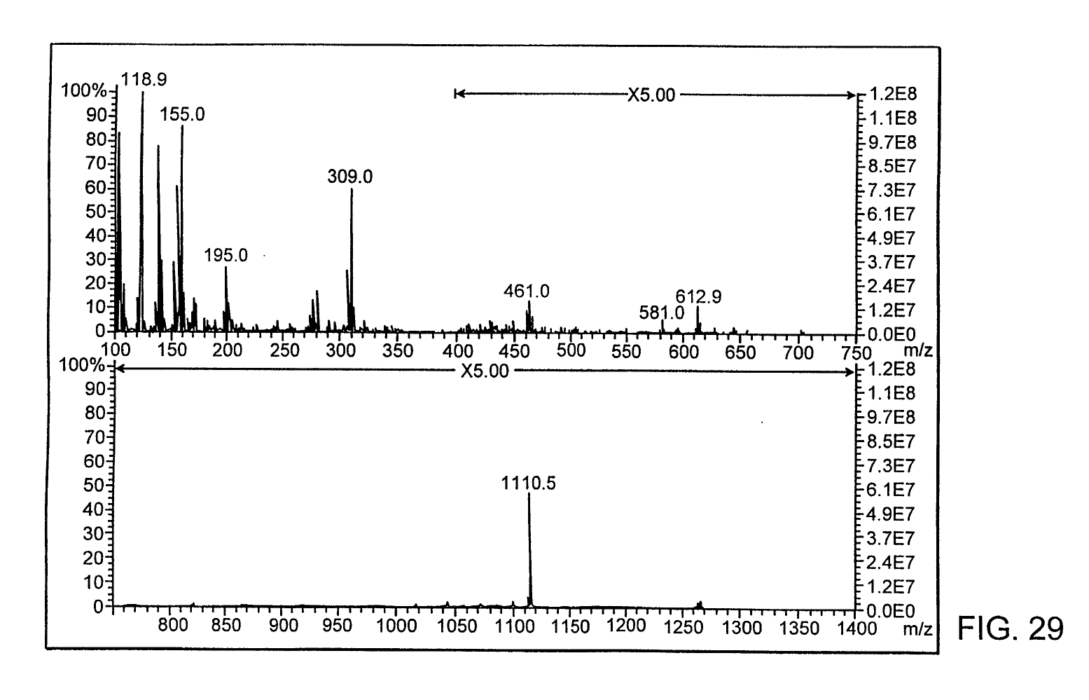

What is claimed is:

1. The compound Gln-Didemnin B.

2. A pharmaceutical composition comprising the compound Gln-Didemnin B and an optional pharmaceutically acceptable excipient, diluent or carrier.

3. The compound Cbz-Gln-Didemnin B.

4. A pharmaceutical composition comprising the compound Cbz-Gln-Didemnin B and an optional pharmaceutically acceptable excipient, diluent or carrier.

5. The compound pGlu-Didemnin B.

6. A pharmaceutical composition comprising the compound pGlu-Didemnin B and an optional pharmaceutically acceptable excipient, diluent or carrier.

7. The compound Cbz-pGlu-Didemnin B.

8. A pharmaceutical composition comprising the compound Cbz-pGlu-Didemnin B and an optional pharmaceutically acceptable excipient, diluent or carrier.

9. The compound Gln[GlnIst²]-Didemnin B.

10. A pharmaceutical composition comprising the compound Gln[GlnIst²]-Didemnin B and an optional pharmaceutically acceptable excipient, diluent or carrier.

11. The compound Cbz-Gln[Cbz-GlnIst²]-Didemnin B.

12. A pharmaceutical composition comprising the compound Cbz-Gln[ Cbz-GlnIst²]-Didemnin B and an optional pharmaceutically acceptable excipient, diluent or carrier.

13. A synthetic process for the preparation of Didemnin M comprising the step of coupling the pyroglutaminylglutamine compound (7) with Didemnin B to afford Didemnin M.

14. A synthetic process for the preparation of Didemnin M comprising the steps of:
(a) coupling o-benzyllactylproline (8) with Didemnin A to yield a protected derivative (15);
(b) hydrogenation of derivative (15) to afford Didemnin B; and
(c) coupling the pyroglutaminylglutamine compound (7) with Didemnin B to afford Didemnin M.

15. A method of treating mammalian neoplastic tumors comprising administering to a mammal in need of such treatment, an effective amount of a pharmaceutical composition defined in claim 2, 4, 6, 8, 10 or 12.

16. A method of treating mammalian RNA or DNA viral infections comprising administering to a mammal in need of such treatment, an effective amount of a pharmaceutical composition defined in claim 2, 4, 6, 8, 10 or 12.

17. A method of treating mammalian bacterial infections comprising administering to a mammal in need of such treatment, an effective amount of a pharmaceutical composition defined in claim 2, 4, 6, 8, 10 or 12.

18. A method of treating mammalian fungal infections comprising administering to a mammal in need of such treatment, an effective amount of a pharmaceutical composition defined in claim 2, 4, 6, 8, 10 or 12.

19. A method of promoting immunosuppression in mammals comprising administering to a mammal in need of such treatment, an effective amount of a pharmaceutical composition defined in claim 2, 4, 6, 8, 10 or 12.
